(19) United States
(12) Patent Application Publication
(10) Pub. No.: US 2009/0124920 A1
(43) Pub. Date: May 14, 2009

(54) BIOFEEDBACK DEVICES, SYSTEMS AND METHOD

(76) Inventors: Kip Errett Patterson, Raleigh, NC (US); Rex Hartzell, Topeka, KS (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 11/937,705

(22) Filed: Nov. 9, 2007

(51) Int. Cl. A61B 5/0482

(52) U.S. Cl. 600/544

(57) ABSTRACT

Biofeedback devices, systems, and methods for training enhanced performance are provided. A subject's EEG activity can be measured and analyzed, and a feedback signal can provide ongoing, continuous information regarding the subject's success in maintaining the brain's activity within a predetermined, desired state of enhanced performance. There are specific EEG patterns that maintain states of enhanced performance. The differential reduction and enlargement of the amplitudes of the various frequencies at specific sites that produce states of enhanced performance are measured. For example, enhanced performance can be produced when theta activity (4-8 Hz) and low-alpha activity (8-10 Hz) are maintained below a low-activity threshold while maintaining gamma activity (38-42 Hz) above a performance threshold amplitude (e.g., 0.5 microvolts). Additionally, positive feedback can also be withheld unless the subject maintains high-alpha activity (11-13 Hz) above a staging threshold value. The threshold settings used can be varied to target greater degrees of proficiency.

### TECHNICAL FIELD

The subject matter described herein relates generally to the field of devices, systems and methods for biofeedback. More particularly, the subject matter described herein relates to neurofeedback in which sensory stimuli can be presented to a subject based upon predetermined conditions to encourage brain function generally associated with attention and focus.

### BACKGROUND

Enhanced performance and proficiency of many behaviors can be achieved as the result of specific physiological states within the brain. Sometimes referred to as being “on fire,” “in the flow,” or “in the zone,” these physiological states can be defined by the oscillatory patterns of brain neurons. In fact, a wide range of psychological and physiological states can be linked to a corresponding range of specific patterns of brain activity. Very low frequencies of brain activity referred to as delta activity (e.g., about 0-4 Hz) are generally thought to be correlated to a state of deep sleep. Frequencies in the theta range (e.g., about 4-8 Hz) are often associated with drowsiness or deep relaxation. Alpha activity (e.g., frequencies about 8-13 Hz) is considered characteristic of a relaxed yet alert state. Beta activity (e.g., frequencies about 13-26 Hz) is often associated with active, busy, or anxious thinking. Finally, gamma activity (e.g., frequencies above about 26 Hz, particularly about 38-42 Hz) is thought to be involved in focused attention and concentration.

The oscillatory patterns of brain neurons are detectable at the scalp as electroencephalographic (EEG) activity. Accordingly, measurements of the frequency of brain activity of a subject can be obtained non-invasively and can then be used to identify the psychological state of the subject. By providing these measurements back to the subject, the subject can become aware of and learn to evoke specific mental states. This process whereby EEG activity is presented to the subject by the means of meaningful sensory stimuli, such as lights, sounds, and/or computerized displays is called biofeedback or, more specifically, neurofeedback. Despite the development of some forms of presented to the subject by the means of meaningful sensory stimuli, such as lights, sounds, and/or computerized displays is called biofeedback or, more specifically, neurofeedback. Despite the development of some forms of neurofeedback, though, there exists a need for devices, systems, and methods for eliciting high levels of attention and focus through neurofeedback.

### SUMMARY

In accordance with this disclosure, devices, systems, and methods are provided for providing neurofeedback to a subject based upon predetermined conditions to encourage brain function generally associated with attention and focus.

According to one aspect the subject matter disclosed herein can include a method for evaluating a subject for enhanced performance. First, the electroencephalographic activity of a subject can be monitored. Then, the amplitudes of electroencephalographic activity in bandwidths having frequencies associated with theta activity, low-alpha activity, and gamma activity can be identified. The theta activity, low-alpha activity, and gamma activity can be compared to a desired pattern of electroencephalographic activity corresponding to enhanced performance. Finally, one or more feedback signals can be operated when the theta activity, low-alpha activity, and gamma activity match the desired pattern of electroencephalographic activity corresponding to enhanced performance.

According to another aspect, the subject matter disclosed herein can include a method for evaluating a subject for enhanced performance. A low-activity threshold amplitude and a performance threshold amplitude can be defined. Electroencephalographic activity of a subject can be monitored. The electroencephalographic activity can be filtered into one or more discrete bandwidths. The amplitudes of the discrete bandwidths having frequencies of 4-8 Hz, 8-10 Hz, and 38-42 Hz can be measured. The amplitude of 4-8 Hz and 8-10 Hz activity can be compared to the low-activity threshold amplitude, and the amplitude of 38-42 Hz activity can be compared to the performance threshold amplitude. A positive feedback signal that is detectable by the subject can then be activated when the 4-8 Hz and 8-10 Hz activities are less than the low-activity threshold amplitude while the 38-42 Hz activity is greater than the performance threshold amplitude.

According to yet another aspect, the subject matter disclosed herein can include an apparatus or device for evaluating a subject for enhanced performance. The apparatus can include one or more sensors for monitoring electroencephalographic activity of a subject. Also, a filter can be included for identifying the electroencephalographic activity in discrete bandwidths having frequencies associated with theta activity, low-alpha activity, and gamma activity. A signal analyzer can compare the amplitude of theta activity, low-alpha activity, and gamma activity to a desired pattern of electroencephalographic activity. One or more signals can be included for providing feedback to the subject indicating whether the subject is exhibiting the desired pattern of electroencephalographic activity.

### BRIEF DESCRIPTION OF THE DRAWINGS

Embodiments of the subject matter described herein will now be explained with reference to the accompanying drawings, of which:

FIG. 1 is a block diagram illustrating aspects of an embodiment of the present subject matter;

FIG. 2 is a block diagram illustrating aspects of another embodiment of the present subject matter;

FIG. 3 is a front plan view of a device of the present subject matter;

FIG. 4 is an example of an electrode placement chart for use with a subject in accordance with the present subject matter; and

FIG. 5 is a schematic circuit and component diagram of a device in accordance with an embodiment of the present subject matter.

### DETAILED DESCRIPTION

Reference will now be made in detail to possible embodiments of the present subject matter, one or more examples of which are shown in the figures. Each example is provided to explain the subject matter and not as a limitation. In fact, features illustrated or described as part of one embodiment can be used in another embodiment to yield still a further embodiment. It is intended that the present subject matter cover such modifications and variations.

There are specific EEG patterns that maintain states of enhanced performance. Although various frequencies of neuron membrane oscillation exist more or less continuously throughout the brain, including those discussed in more detail hereinbelow, it is the differential reduction and enlargement of the amplitudes of the various frequencies at specific sites that produce states of enhanced performance. For example, enhanced performance can be produced when the amplitude of brain activity having frequencies of approximately 38-42 Hz (a sub-band of the gamma activity band) is maintained at an elevated level as compared to activity in other frequencies. More specifically, enhanced performance can be produced when the amplitude of activity having frequencies of approximately 4-8 Hz (frequently understood as theta activity) and approximately 8-10 Hz (a sub-band on the low end of the typical alpha activity band) is maintained below a specified low-activity threshold while simultaneously maintaining brain activity having a frequency of approximately 38-42 Hz above a minimum high-activity, performance threshold amplitude.

In addition to the achievement of this pattern of brain activity, the production of the state of enhanced performance can be further developed by maintaining EEG activity having a frequency of approximately 11-13 Hz (a sub-band on the high end of the typical alpha frequency band) above a staging threshold prior to the reduction of theta and low-alpha activity and the elevation of gamma activity above a minimum performance threshold. This additional sequential pattern of activation involves a greater degree of proficiency because the subject must exhibit this preparatory level of brain activity prior to exhibiting the pattern of activity corresponding to enhanced performance. The extra step of activating the high-alpha activity is thought to prepare the subject to achieve a state of focus and attention, thus increasing the subject's proficiency at the activity being trained.

The specific thresholds required of each of the bandwidths can vary depending on the degree of enhanced performance sought or the skill of the subject in attaining the desired mental state. For example, the low-activity threshold can be, for example, as high as about 30 microvolts or more. In contrast, the performance threshold amplitude can be comparatively small, generally about 0.5 microvolts, and still require the subject to produce the level of focus and attention desired for enhanced performance. It is thus not necessarily the levels of activity of each category of brain activity, but the differential pattern of activity which determines and creates states of enhanced performance.

That being said, within the differential pattern of activity, greater degrees of proficiency can be positively correlated with lower thresholds for the theta and low-alpha activity bandwidths. For instance, performance below a 4 microvolt low-activity threshold is generally more proficient than performance below an 8 microvolt low-activity threshold for the same individual within the same time frame. The training of reduced theta and low-alpha activity, therefore, can include an easily adjusted low-activity threshold setting. As the subject becomes more adept at maintaining low-activity frequencies below a given low-activity threshold, the threshold can thus be adjusted to target greater levels of proficiency. Similarly, the comparatively low performance threshold amplitude setting (e.g., typically about 0.5 microvolts) for activity in the gamma activity bandwidth is provided as an example of a minimum threshold. As with progressively decreasing levels of the theta and low-alpha activity, increasing levels of activity in the gamma band can correspond to increased levels of focus and attention.

Still, there is a limit to the reliability of these correlations. EEG activity is highly variable and can be dependent upon circadian rhythms, metabolism, environmental variables, and hydration, among other factors. A subject might train one day to a threshold setting of 6 microvolts and another day to a threshold setting of 4 microvolts with equal training benefit. Accordingly, reaching absolute minimum low-activity threshold settings is not necessary because a reduction in the low-activity threshold provides only incremental improvements in performance in comparison to the effect of differential amplitude patterns of the specified EEG bandwidths. Likewise, with respect to gamma activity, because it is the differential amplitude patterns that play more of a factor in driving the achievement of states of enhanced performance, maintaining gamma activity above a defined minimum performance threshold is all that is necessary.

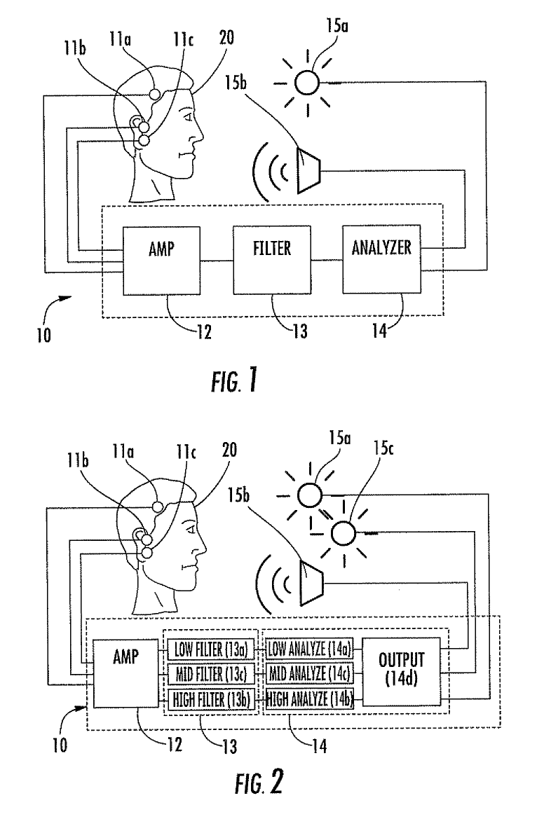

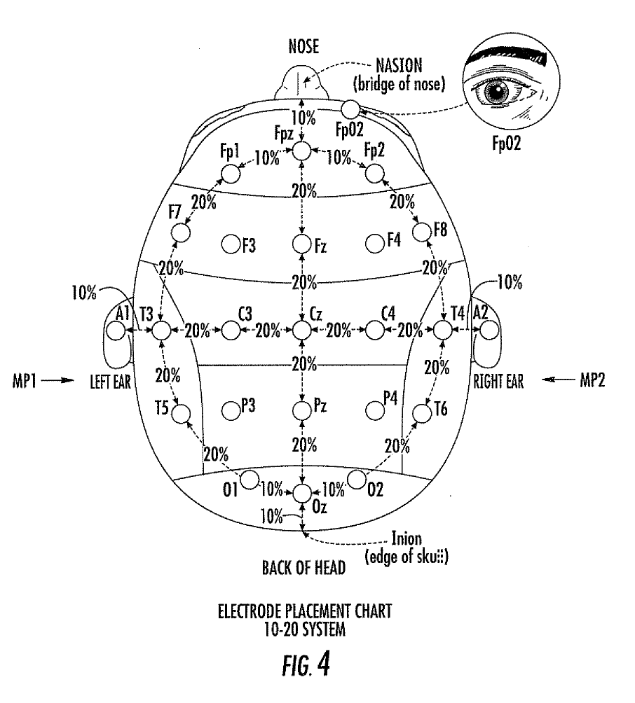

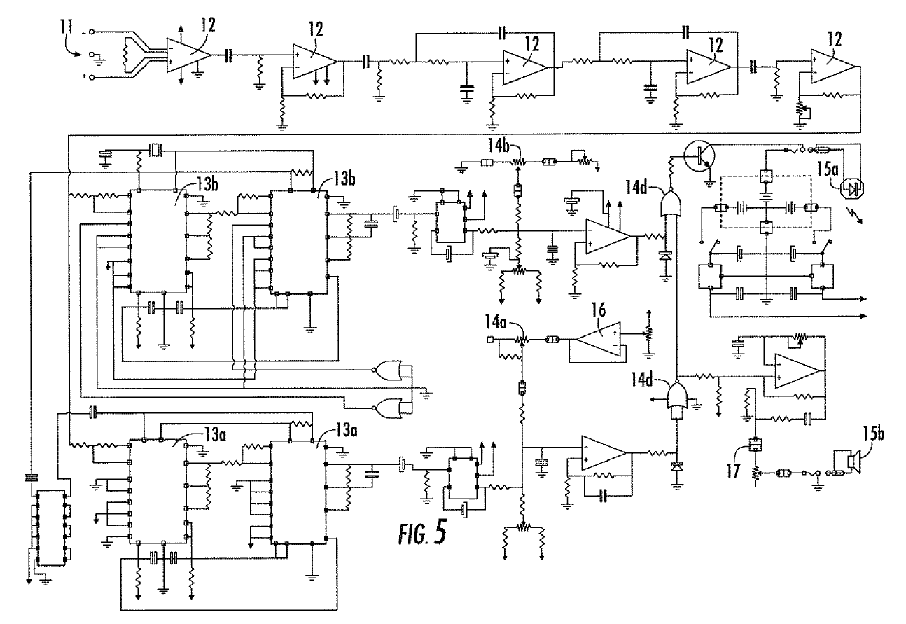

To measure a subject's EEG activity, a biofeedback training device generally designated 10 in FIG. 1 can be provided. As shown in FIGS. 1-3, one or more EEG sensors 11 (e.g., electrodes) can be positioned on the scalp of a subject 20 to detect the EEG activity. The sites on the scalp from which the EEG is recorded can be specifically selected to provide consistency in comparing the activity of multiple subjects or tracking changes in the activity of a single subject over time. In addition, the specific sites selected can play a role in focusing the neurofeedback training. For example, using points from the International 10-20 System of electrode placements depicted in FIG. 4, placements along the temporal lobes F8 to T4 and F7 to T3 benefit emotional control during enhanced performance. Placements along the sensory-motor strips of the brain, CZ to T3 and CZ to T4, benefit psychomotor control.

The role of the cerebellum is important in the performance of any motor act, and recent research has implicated the cerebellum in a much broader range of human mental activity than was previously believed to be the case.

The present method can be used to influence the activity of the cerebellum by placing the EEG sensors 11 of the biofeedback training device 10 over anatomical sites corresponding to the cerebellum. In particular, the site termed the “mastoid process” is an easily identified bony protuberance located posterior to the ear which is not described in either the International 10-20 or 10-10 Systems. It is located just behind the external acoustic meatus, and lateral to the styloid process. Stated otherwise, the mastoid process is generally located just below and rearward of the ear canal. It is, however, directly above the location of the cerebellum.

To be congruent with the International 10-20 and 10-10 Systems of nomenclature, the left mastoid process is hereafter referred to as MP1 and the right mastoid process is referred to as MP2. These points are located generally as identified in FIG. 4. Training within the currently described method at MP1 is thought to help the subject to improve the acquisition of motor behavior patterns whereas training at MP2 is thought to help the subject to consolidate already acquired behavior patterns and produce a sense of “physical confidence.” Training sessions at MP1 and MP2 can thereby be integrated within a larger training program for skill acquisition, skill enhancement, and/or skill consolidation.

Referring again to FIGS. 1-3, to obtain a clear measurement of brain activity, the one or more EEG sensors 11 can include one or more “live” signal electrodes 11a positioned on the head of the subject 10. A reference electrode 11b and a “ground” electrode 11c (i.e., second reference) can be positioned at an EEG neutral location to provide a means by which to filter out any noise. Because the ear is considered EEG neutral (i.e., no brain activity is detected), the ear lobes can be used for a reference base, thereby producing “cleaner” brain wave activity measurements.

Monopolar EEG recording involves one signal electrode 11a being positioned on the head of the subject 20. Monopolar recording is generally considered sufficient to obtain useful EEG measurements, and it is even preferred by some because of the precision of the feedback for a specific training site. Because only one signal electrode 11a is used to obtain the EEG signal, only the activity at the site of that signal electrode 11a is monitored, so there is no risk of disparate multiple signals complicating the analysis of the subject's brain activity. When using a three-electrode system in this arrangement, both a reference electrode 11b and a ground electrode 11c can be provided at an EEG neutral position to serve as reference measurements. For example, in the 10-20 System, training with a live electrode 11a at position C4 can be coupled with a reference electrode 11b on the right ear at position A2 and a “ground” electrode 11c on the left ear at position A1. Similarly, to target the cerebellum, the live electrode 11a can be placed at MP1, the reference electrode 11b at A2, and the “ground” electrode 11c at A1.

Alternatively, bipolar EEG recording can be used as well, incorporating two signal electrodes on the head. When using a three-electrode system in this configuration, both the signal electrode 11a and the reference electrode 11b can be positioned at points on the scalp of the subject 20 to obtain “live” readings of EEG activity, with the ground electrode 11c remaining at an EEG neutral location as the reference signal. For example, in the 10-20 System, a live electrode 11a can be placed at C4, a reference electrode 11b at T4, and a “ground” electrode 11c on the left ear at A1. Similarly, the live electrode 11a can be placed at MP2, the reference electrode 11b connected at T6, and the “ground” electrode 11c connected at A1. One advantage of bipolar recording is the ability to monitor the coordination of activity in multiple sections of the brain. For instance, the placement of the signal electrodes 11a, 11b at inter-hemispheric sites can be useful for training the smooth coordination of alternating behavior patterns, such as changing the pivot points during a golf swing from the back hip to the front hip. For example, in the 10-20 System, the live electrode 11a can be placed at C3 and the reference electrode 11b at C4 (or vice-versa) to target this kind of activity. In another configuration, the two signal electrodes 11a, 11b can be positioned on the same side of the head to isolate the function of a single muscle group. To avoid the production of a cluttered signal by combining different signals from multiple points, the EEG sensors can be positioned at recording sites that are close together.

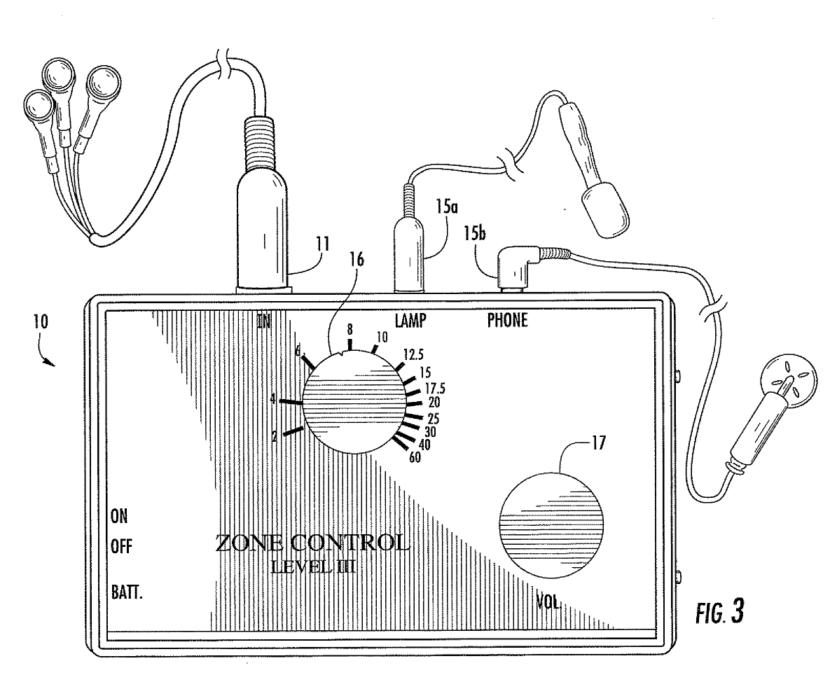

The EEG signals acquired by the biofeedback training device 10 can then be analyzed to determine the amplitude of EEG activity in each of the bandwidths of interest. For instance, one or more filters 13 (FIGS. 1 and 2) can be provided to identify activity in discrete frequencies or bands of frequencies within the raw EEG signal. Specifically, the EEG signal can be processed so that activity at one or more frequencies of about 4-8 Hz (theta), about 8-10 Hz (low-alpha), about 11-13 Hz (optional high-alpha measurement), and about 38-42 Hz (gamma) can be individually identified. Further, one or more signal amplifiers 12 (FIGS. 1 and 2) can be provided to make the individual frequencies more easily identifiable, and thus make the discrete bands more easily discernable. The discrete bands can then be individually analyzed to determine the amplitude of activity in each band. In particular, one or more signal analyzers 14 (FIGS. 1 and 2) can compare each band to the relevant threshold amplitude corresponding to enhanced performance. The one or more signal analyzers 14 can include or be, for example, a microcomputer, a digital circuit, or a circuit board having a series of logic circuits. In addition, one or more threshold control mechanisms 16, such as a knob or dial, can be provided to allow the operator of the device to set one or more of the threshold amplitude settings. For example, a control knob can be provided for defining the low-activity threshold amplitude below which the subject 20 must maintain his or her theta and low-alpha activity levels.

After the EEG signal is analyzed, one or more feedback signals can be presented to the subject 20. During neurofeedback training, the feedback signal can provide ongoing, continuous information regarding the subject's success in maintaining the brain's activity within the state of enhanced performance. For instance, a positive feedback signal 15a can be provided to denote that the brain is in a state of enhanced performance. Specifically, positive signal 15a can operate to turn on when gamma activity is elevated as compared to other levels of activity. More specifically, positive signal 15a can be activated when the theta, low-alpha, high-alpha, and gamma activity levels match the desired pattern of electroencephalographic activity corresponding to enhanced performance. In this way, the subject 20 can be made aware of his or her achievement of a predetermined or desired pattern of brain activity. The subject 20 is thus provided with external positive reinforcement for achieving a state of enhanced performance.

Alternatively, one or more negative feedback signals 15b can be provided to denote the absence of a state of enhanced performance. Negative signals 15b can operate to turn off when the desired pattern of activity is exhibited. That is to say, negative signal 15b can be configured to be on only when the desired pattern of activity is not present. When subject 20 generates theta and/or low-alpha activity above the low-activity threshold, or when subject 20 does not generate gamma activity above the performance threshold, negative signal 15b can be provided. Negative signal 15b can thus be used to deter undesirable activity, or at least make subject 20 aware that he or she is not exhibiting the predetermined, desired pattern of brain activity. In this regard, negative signal 15b can be designed to be unpleasant or obnoxious such that subject 20 is further motivated to achieve a state of enhanced performance to turn off the negative feedback.

Whether using a positive feedback signal 15a, a negative feedback signal 15b, or both, devices, systems, and methods in accordance with this disclosure for biofeedback can teach subject 20 to exhibit the desired levels of focus and concentration during the performance of an activity being trained.

There are also additional means by which a feedback signal provided by the biofeedback training device 10 can be designed to optimize the effectiveness of the neurofeedback training. For example, a feedback signal can be configured so that the time between the recording of the brain activity and the presentation of the feedback signal to subject 20 is not greater than about 0.5 to 0.75 seconds. In this way, the feedback signal is more closely correlated with the EEG patterns measured to enable subject 20 to more effectively recognize his or her mental state. Further, a feedback signal can be designed to be easily detected, simple in format, and/or able to be consigned to peripheral awareness so that the operation of the feedback signal does not interfere substantially with the performance of the activity under training.

In particular, a feedback signal can be one or more visual signals, such as a visible light from a light-emitting diode attached to subject 20 within the subject's field of vision. Alternatively, a feedback signal can be an auditory signal, such as an audible tone delivered by means of an ear piece to subject 20, or it can be a vibrotactile signal, such as a small vibration generator in communication with subject 20. More detailed or complex signals can be used, but increased complexity can potentially be distracting, causing subject 20 to divert his or her attention from the task under training. Consequently, it is thought that unobtrusive signals that can be perceived peripherally are well-suited for training enhanced performance of a specified task.

A signal level control 17 (e.g., control knob or dial) can be provided to maintain the output of a feedback signal at a level that is perceptible but not so apparent that the signal interferes with the subject's concentration. For example, for an auditory feedback signal, a volume control can be provided so that subject 20 can adjust the volume to a level that is audible but is not so loud that subject 20 is distracted.

The biofeedback training device 10 can further be designed to be portable such that subject 20 can wear it without hindrance, especially during the training of specific tasks. A biofeedback training device 10 that is lightweight and can be easily clipped or otherwise secured to subject 20 more readily allows subject 20 to train his or her brain activity for enhanced performance within the environment of the activity being trained. In contrast, a biofeedback training device 10 that relies on a connection to a separate, external computer to perform the analysis restricts the ability of subject 20 to move freely. For example, if subject 20 wishes to train to achieve a state of enhanced performance as he or she plays a round of golf, he or she can only do so if the training device is portable and lightweight so as to not impose a burden as subject 20 moves about the course. Of course, training for enhanced performance in “virtual” settings, such as in an office, indoor training facility, or seminar setting, by imagining performance scenarios and visualizing desired outcomes can be beneficial. Nevertheless, training in the real environment, such as on a golf course, at a bowling alley, or on a target range, is considered by some to be more effective in achieving ultimate performance. Accordingly, a biofeedback training device 10 that is small and portable enables subject 20 to realize the added benefit of training his or her brain for enhanced performance in an immersive setting.

The devices, systems and methods for neurofeedback disclosed herein can be used to train greater proficiency in a wide range of behavior patterns because the state of enhanced performance can be a “content-free” state. In other words, any activity that can be influenced by increased focus and attention can be trained to a greater degree of proficiency by increasing gamma activity as compared to other frequencies or, more particularly, by reducing the theta and low-alpha bandwidth amplitude while simultaneously maintaining the gamma bandwidth activity above a performance threshold amplitude. Accordingly, the neurofeedback methodology disclosed above can be used to train subjects, such as subject 20, for enhanced performance in golf, bowling, shooting, chess or any activity in which focus and concentration are relevant to successful performance.

The disclosed subject matter can also be used to detect behavior sequences that are less than optimally proficient. This becomes obvious to subject 20 (and to an instructor, should one be present) via the differential activity of the feedback signals. For example, the disclosed subject matter can be used to train a golfer to maintain a desired level of concentration throughout the performance of his or her golf swing, which can be a complete swing such as with an iron or a wood or even a putting stroke. Subject 20 can be required to activate a positive feedback signal 15a while standing stationary as he or she addresses a golf tee shot and keep the positive signal 15a on through the entire sequence of his or her swing. Thus, even if subject 20 maintains the appropriate brain activity during the initial portions of his golf swing, if subject 20 then loses focus or has his or her attention diverted at the farthest extent of the backswing of the golf club, the positive signal 15a goes off and the negative signal 15b comes on.

Feedback such as this can indicate both that a less than optimal behavior is being performed and the specific position in a sequence of an activity such as a golf swing that the breakdown in concentration occurred. By observing the point at which the feedback signals reversed from positive to negative, subject 20 (and instructor) can identify problem areas in the golfer's swing. Further, by using a biofeedback training device 10 that minimizes the lag time between brain events and the feedback signals 15a, 15b (e.g., less than about 0.5-0.75 seconds), the precise location of the problem can be more effectively pinpointed. Subject 20 can then self-correct (or receive corrective instruction) until he or she is able to maintain feedback signals 15a, 15b in the positive state during the entire sequence of the golf swing.

The devices, systems and methods for neurofeedback disclosed herein can have application to proficiency training as well as to instructional correction of behavior sequences and the creation of more efficient, purposeful behavior patterns.

With reference to an exemplary configuration of the devices, system and method for neurofeedback depicted in FIGS. 1-3 and 5, subject 20 can be connected to a portable biofeedback training device 10 and can begin training at a low-activity threshold of 30 microvolts defined by a threshold control mechanism 16. Electrodes 11a, 11b, and 11c connected to subject 20 can supply the EEG activity to one or more signal amplifiers 12. The amplified signal can then be supplied to one or more bandpass filters 13, which provide isolated signals for the EEG activity bands of interest. For instance, a low-activity filter 13a can isolate EEG activity in the theta and low-alpha bandwidths (e.g., 4-10 Hz) and a performance activity filter 13b can isolate activity in the gamma bandwidth (e.g., 38-42 Hz).

The filtered signals can then be supplied to one or more signal analyzers 14. For instance, a low-activity analyzer 14a can receive a filtered signal from the low-activity filter 13a and compare it to the low-activity threshold defined by threshold control mechanism 16. A performance activity analyzer 14b can receive a filtered signal from the performance activity filter 13b and compare it to the performance threshold. An analyzer output control 14d can then use these comparisons to determine which of the one or more feedback signals to activate.

When subject 20 produces elevated gamma (38-42 Hz) activity as compared to other frequencies, biofeedback training device 10 can turn on a light (positive signal 15a) and turn off a tone (negative signal 15b). More specifically, biofeedback training device 10 can be configured to only turn on a light and turn off a tone when theta (4-8 Hz) and low-alpha (8-10 Hz) activity is maintained below the low-activity threshold amplitude (e.g., 30 microvolts) while simultaneously producing gamma (38-42 Hz) activity above the performance threshold amplitude (e.g., 0.5 microvolts). Once subject 20 gains skill in consciously controlling their mental state such that positive signal 15a stays on and negative signal 15b stays off, threshold control mechanism 16 can be adjusted so that the setting for 4-8 and 8-10 Hz can be reduced (e.g., to 25 microvolts) to train subject 20 to achieve even greater proficiency. Training can proceed in similar fashion, adjusting the low-activity threshold for increasing skill, until subject 20 reaches the lowest threshold setting possible for that day and that time of day.

As noted above, an additional step of activating high-alpha activity can be required prior to the decrease of theta and low-alpha activity and the increase of gamma activity. Training can proceed in a similar or the same fashion as above, with the signal supplied by the one or more amplifiers 12 received by a staging activity filter 13c, which isolates EEG activity in the high-alpha bandwidth (e.g., 11-13 Hz). A staging activity analyzer 14c can then compare the filtered signal to an adjustable threshold. This further comparison is supplied to the output control 14d. In this configuration, subject 20 receives an unobtrusive, ongoing staging feedback signal 15c for increasing 11-13 Hz activity (high-alpha) above a staging threshold. The separate staging signal 15c can be provided to subject 20 to indicate that the subject 20 is mentally primed for enhanced performance.

To distinguish the various signals presented in this multi-stage sequence, staging signal 15c can be different than the positive and negative signals 15a, 15b so that it can be identified distinctly from the other signals. For example, staging signal 15c can be provided as a yellow light, positive signal 15a can be provided as a green light, and negative signal 11b can be provided as a tone.

The complete sequence of differential activation can then be to: (1) activate staging signal 15c by increasing the amplitude of 11-13 Hz brain activity above a defined staging threshold; and (2) deactivate negative signal 15b and/or activate positive signal 15a by decreasing the amplitude of 4-8 and 8-10 Hz brain activity below a defined low-activity threshold while simultaneously maintaining the amplitude of 38-42 Hz activity above a minimum performance threshold. Again, the various thresholds can be set to target varying degrees of enhanced performance, but it is the differential pattern of activation of the various bandwidths that determines the presence or absence of states of enhanced performance.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for evaluating a subject for enhanced performance, comprising:
(a) monitoring electroencephalographic activity of a subject;
(b) identifying amplitudes of electroencephalographic activity in bandwidths having frequencies associated with theta activity, low-alpha activity, and gamma activity;
(c) comparing the theta activity, low-alpha activity, and gamma activity to a desired pattern of electroencephalographic activity corresponding to enhanced performance; and
(d) operating one or more feedback signals in response to the theta activity, low-alpha activity, and gamma activity being within the desired pattern of electroencephalographic activity.

2. A method for evaluating a subject for enhanced performance according to claim 1, further comprising:
(a) defining a low-activity threshold amplitude; and
(b) defining a performance threshold amplitude;
(c) wherein the desired pattern of electroencephalographic activity corresponding to enhanced performance comprises:
(i) the theta activity and low-alpha activity having an amplitude below the low-activity threshold amplitude; and
(ii) the gamma activity having an amplitude above the performance threshold amplitude.

3. A method for evaluating a subject for enhanced performance according to claim 2, wherein the performance threshold amplitude is about 0.5 microvolts.

4. A method for evaluating a subject for enhanced performance according to claim 1, wherein monitoring is performed by placing electrodes on the head of the subject at one or more locations specified by the international 10-20 system.

5. A method for evaluating a subject for enhanced performance according to claim 1 wherein monitoring comprises monitoring electroencephalographic activity from the cerebellum of a subject.

6. A method for evaluating a subject for enhanced performance according to claim 5, wherein monitoring electroencephalographic activity from the cerebellum of a subject is performed by placing electrodes at least one mastoid process of the subject.

7. A method for evaluating a subject for enhanced performance according to claim 1, wherein operating one or more feedback signals is performed within about 0.75 seconds of identifying the amplitudes of electroencephalographic activity.

8. A method for evaluating a subject for enhanced performance according to claim 7, wherein operating one or more feedback signals is performed within about 0.5 seconds of identifying the amplitudes of electroencephalographic activity.

9. A method for evaluating a subject for enhanced performance according to claim 1, wherein the step of operating one or more feedback signals comprises activating a positive signal detectable by the subject when the theta activity, low-alpha activity, and gamma activity is within the desired pattern of electroencephalographic activity.

10. A method for evaluating a subject for enhanced performance according to claim 9, wherein the step of activating a positive signal comprises activating a visible light.

11. A method for evaluating a subject for enhanced performance according to claim 1, wherein the step of operating one or more feedback signals comprises deactivating a negative signal detectable by the subject when the theta activity, low-alpha activity, and gamma activity is within the desired pattern of electroencephalographic activity.

12. A method for evaluating a subject for enhanced performance according to claim 11, wherein the step of deactivating a negative signal comprises deactivating an audible tone.

13. A method for evaluating a subject for enhanced performance according to claim 1, further comprising:
(a) identifying amplitude of electroencephalographic activity in bandwidths having frequencies associated with high-alpha activity;
(b) comparing the high-alpha activity to a desired level of high-alpha activity; and
(c) activating one or more staging signals to the subject when the subject exhibits the desired level of high-alpha activity.

14. A method for evaluating a subject for enhanced performance according to claim 13, wherein activating one or more staging signals is performed when the subject exhibits the desired level of high-alpha activity prior to exhibiting the desired pattern of electroencephalographic activity.

15. A method for evaluating a subject for enhanced performance according to claim 13, further comprising:
(a) defining a staging threshold amplitude;
(b) wherein comparing the high-alpha activity to a desired level of high-alpha activity comprises comparing the high-alpha activity to the staging threshold amplitude; and
(c) wherein activating one or more staging signals comprises activating a staging signal detectable by the subject when the high-alpha activity is greater than the staging threshold amplitude.

16. A method for evaluating a subject for enhanced performance according to claim 13, wherein the step of activating one or more staging signals comprises activating a visible light.

17. A method for evaluating a subject for enhanced performance, comprising:
(a) defining a low-activity threshold amplitude and a performance threshold amplitude;
(b) monitoring electroencephalographic activity of a subject;
(c) filtering the electroencephalographic activity into one or more discrete bandwidth;
(d) measuring amplitudes of the one or more discrete bandwidth having frequencies of about 4-8 Hz, 8-10 Hz, and 38-42 Hz;
(e) comparing the amplitude of 4-8 Hz and 8-10 Hz activities to the low-activity threshold amplitude;
(f) comparing the amplitude of 38-42 Hz activity to the performance threshold amplitude; and
(g) activating a positive feedback signal detectable by the subject when the 4-8 Hz and 8-10 Hz activities are less than the low-activity threshold amplitude while the 38-42 Hz activity is greater than the performance threshold amplitude.

18. A method for evaluating a subject for enhanced performance according to claim 17, further comprising:
(a) defining a staging threshold amplitude;
(b) measuring amplitude of the one or more discrete bandwidth having frequencies of about 11-13 Hz;
(c) comparing the amplitude of 11-13 Hz activity to the staging threshold amplitude; and
(d) activating a staging signal when the 11-13 Hz activity is greater than the staging threshold amplitude prior to activating a positive feedback signal.

19. A device for evaluating a subject for enhanced performance, comprising:
(a) one or more sensors for monitoring electroencephalographic activity of a subject,
(b) a filter for identifying the electroencephalographic activity in one or more discrete bandwidth having frequencies associated with theta activity, low-alpha activity, and gamma activity;
(c) a signal analyzer for comparing amplitude of theta activity, low-alpha activity, and gamma activity to a desired pattern of electroencephalographic activity; and
(d) one or more signals for providing feedback to the subject for indicating whether the subject is exhibiting the desired pattern of electroencephalographic activity.

20. The device for evaluating a subject for enhanced performance according to claim 19, further comprising one or more signal amplifiers for amplifying the electroencephalographic activity before it is provided to the filter.

21. The device for evaluating a subject for enhanced performance according to claim 19, wherein the signal analyzer comprises:
(a) a low-activity analyzer for comparing amplitude of theta activity and low-alpha activity to a low-activity threshold amplitude; and
(b) a performance analyzer for comparing amplitude of gamma activity to a performance threshold amplitude.

22. The device for evaluating a subject for enhanced performance according to claim 21, further comprising a low-activity threshold control mechanism for defining the low-activity threshold.

23. A method for evaluating a subject for enhanced performance, comprising:
(a) monitoring electroencephalographic activity of a subject;
(b) identifying amplitude of electroencephalographic activity in bandwidths having frequencies of about 38-42 Hz;
(c) comparing the amplitude of 38-42 Hz activity to a desired pattern of electroencephalographic activity corresponding to enhanced performance; and
(d) operating one or more feedback signals in response to the 38-42 Hz activity being within the desired pattern of electroencephalographic activity.

24. A device for evaluating a subject for enhanced performance, comprising:
(a) one or more sensors for monitoring electroencephalographic activity of a subject;
(b) a filter for identifying the electroencephalographic activity in bandwidths having frequencies of about 38-42 Hz;
(c) a signal analyzer for comparing amplitude of 38-42 Hz activity to a desired pattern of electroencephalographic activity; and
(d) one or more signals for providing feedback to the subject for indicating whether the subject is exhibiting the desired pattern of electroencephalographic activity.
